(19) United States
(12) Patent Application Publication
(10) Pub. No.: US 2022/0213436 A1
(43) Pub. Date: Jul. 7, 2022

(54) BRAIN ORGANOID MACHINE INTERFACE

(71) Applicant: The Regents of the University of California, Oakland, CA (US)

(72) Inventor: ALysson Renato Muotri, San Diego, CA (US)

(21) Appl. No.: 17/609,534

(22) PCT Filed: May 7, 2020
(86) PCT No.: PCT/US2020/031844

§ 371 Date: Nov. 8, 2021

Related U.S. Application Data
(60) Provisional application No. 62/844,700, filed on May 7, 2019.

(51) Int. Cl. C12N 5/079; C12M 1/42

(52) U.S. Cl. CPC C12N 5/0618; C12M 35/02; C12N 2500/38; C12N 2501/115; C12N 2501/13; C12N 2533/90; C12N 2503/04; C12N 2506/02; C12N 2506/45; C12N 2513/00; C12N 2501/727

(57) ABSTRACT

The disclosure provides methods of making and systems comprising a brain organoid operably connected to a controlled device such that the brain organoid controls the controlled device.

### CROSS REFERENCE TO RELATED APPLICATIONS

This application claims priority under 35 U.S.C. § 119 from Provisional Application Ser. No. 62/844,700, filed May 7, 2019, the disclosures of which are incorporated herein by reference.

### TECHNICAL FIELD

The disclosure provides methods of making functional cortical organoids from somatic cells and stem cells and methods of using the organoids to manipulate actions and activities of robotic systems and interfaces between the organoid and the robotic systems.

### BACKGROUND

Learning is a complex cognitive process, shaped by evolution over millions of years and modulated by the nervous system in real time. An essential step towards understanding human cognition is to dissect the fundamental biological mechanisms of learning. During neural maturation, the brain shifts from an experience-independent but activity-dependent network formation at an embryonic stage, to sensory experience-dependent learning after birth. Thus, previous investigations on the biological mechanisms of learning focused on postnatal subjects, ignoring the critical neurodevelopmental period when this transition happens in utero. Brain or cortical organoids, derived from human pluripotent stem cells, can mimic early stages of neurodevelopment in a dish, providing a promising experimental model to dissect the molecular and cellular mechanisms of learning.

### SUMMARY

A cortical organoid model described herein has been developed to capture the aforementioned critical period of early learning. Experiments were performed to manipulate spontaneously generated network activity to investigate the role of experience-independent activity on synapse formation. A closed-loop robotic system was also designed that provides sensorimotor feedback to the cortical organoid when exploring the environment, to evaluate experience-dependent learning with controlled physical variables.

The disclosure provides a brain-organoid machine interface, comprising a brain organoid culture; a sensor comprising a plurality of electrodes that sense voltage change information including amplitude and/or frequency in the brain organoid culture; a processing unit to receive the voltage change information from the sensor, process the voltage change information to produce processed signals, and transmit the processed signals; and a controlled device for receiving the processed signals. In one embodiment, the sensor comprises a multi-electrode array (MEA) to sense voltage change information including amplitude and/or frequency in the brain organoid culture. In another or further embodiment, the sensor is attached to or immediately adjacent to a substrate comprising the brain organoid culture. In still another or further embodiment, the sensor is in contact with the brain organoid culture. In another embodiment, the sensor and processing unit are connected with one or more cables comprising electrical or optically conducting wires. In yet another embodiment, the sensor and processing unit are wirelessly connected. In still another embodiment, the voltage change information comprises one or more of neuron spikes, electrocorticogram signals, local field potential signals, and electroencephalogram signals. In another embodiment, the MEA is patterned. In still yet another embodiment, the controlled device is one or more of the group consisting of a computer, a computer display, a mouse, a cursor, an artificial or prosthetic limb, a robot or robotic device, a computer controlled device, a vehicle, and a communication device or system. In a further embodiment, the controlled device comprises a feed-back system. In yet a further embodiment, the feed-back system comprises signals to deliver a physical, optical or chemical stimuli to the brain organoid culture transmitted from a sensor on the controlled device. In another embodiment, the brain organoid culture is comprised predominantly of proliferative neural progenitor cells (NPCs) that have self-organized into a polarized neuroepithelium-like structure. In another embodiment, the brain organoid culture comprise pyramidally-shaped neurons, dendritic spines and structurally defined synapses. In another embodiment, the brain organoid culture comprises cells differentiated from induced pluripotent stem cells (iPSCs). In a further embodiment, the iPSCs are derived from somatic cells of a subject with a neurodegenerative disease or disorder. In still another or further embodiment, the controlled device responds to the processed signals. In a further embodiment, the responses of the controlled device are used to evaluate neurological activity and/or function of the brain organoid. In still a further embodiment, the controlled device is used to study changes in neurological activity and/or function in the presence and absence of an external stimuli. In yet a further embodiment, the external stimuli is a chemical agent, optical or electrical stimuli, a drug or protein.

### DESCRIPTION OF DRAWINGS

FIG. 1 provides a high level overview of the quadruped robot main parts, with coupled ultrasonic sensors for environmental exploration

FIG. 2 is a simplified schematic overview of the current robotic platform controlled from signals generated by a human brain organoid.

FIG. 3 provides a schematic representation of the closed feedback learning circuitry proposed between the brain cortical organoid signals captured by MEAs and controlled device (e.g., a robot). In the schematic the organoid sends signals to the controlled device to perform a function. As the controlled device functions the controlled device can send signals back to the organoids. For example, the organoid can provide signals that cause a robot to walk, as the robot moves forward it encounters an obstacle and sends a signal back to the organoid; in order to stimulate the brain organoids based on the robot environmental exploration (facing an obstacle), electric/chemical/light stimuli could be applied directly to the cortical organoids. As a consequence, the networks will respond, sending novel signals to the computer that will then instruct the robot to perform a different task (stop, walk back, etc.).

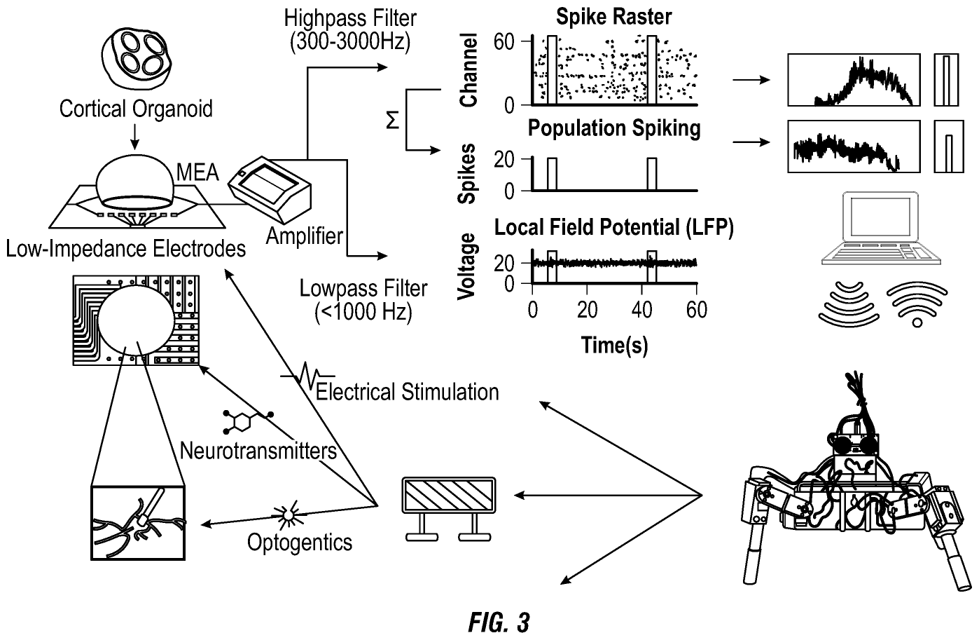

### DETAILED DESCRIPTION

As used herein and in the appended claims, the singular forms “a,” “an,” and “the” include plural referents unless the context clearly dictates otherwise. Thus, for example, reference to “a neurosphere” includes a plurality of such neurospheres and reference to “the organoid” includes reference to one or more organoids and equivalents thereof known to those skilled in the art, and so forth.

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated by reference in full for the purpose of describing and disclosing methodologies that might be used in connection with the description herein. The publications are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

Development of functional human brain networks is an activity-dependent process guided by genetic and molecular programs and shaped by emerging cellular diversity. Neonate neural networks share many features with adult brains, despite the fundamental structural differences. Even though the chronological stages of the human cortical network formation are not well understood, it is suggested that emerging cognitive functions during infancy are a result of different brain regions and environmental cues. However, in uterus development is vital for the establishment of neuronal circuitry and healthy functioning of the brain. The second and third trimester of gestation are when the corticothalamic network is formed via transient connections of the subplate GABAergic neurons and the emergence of synchronized network activity. Thus, early cortical functional maturation follows an independent sensory-input pathway, guided by spontaneous activity and associated with synaptic regulating mechanisms.

As used herein, the term “differentiation” refers to the process where a cell changes from one cell type to another, e.g., a neural progenitor cell (NPC) differentiates into a neuron or glial cell. Differentiation dramatically changes a cell's size, shape, membrane potential, metabolic activity, and responsiveness to signals. These changes are largely due to highly controlled modifications in gene expression.

As used herein, the term “functional cortical organoids” refers to artificially grow, in vitro, miniature organs that contain well-organized neural progenitor cell (NPC) layers and neuronal layers that are further characterized by producing nested oscillatory waves, e.g., oscillatory waves comparable electroencephalography (EEG)-like network activity upon differentiation. The “functional cortical organoids” can be further characterized by the gene expression profiles, electrophysiological features, and other characteristics presented in this disclosure. Compared with cerebral organoids, brain region-specific organoids, like cortical organoids, model individual brain regions of interest and generally result in more uniform and reproducible tissue, providng a platform for quantitative characterization.

As used herein, the term “high throughput screening” refers to a method for scientific experimentation especially used in drug discovery that can rapidly identify active compounds, small molecules, polypeptides, antibodies, biologically active oligonucleotides or genes that modulate a particular biomolecular pathway. High-throughput screening allows a researcher to quickly conduct thousands if not millions of chemical, genetic, or pharmacological tests. Typically, “high throughput screening” is automated by use of robotics, data processing/control software, liquid handling devices, and sensitive detectors.

As used herein, the term “neurodegenerative insult” refers to an action, such as by exposure to a chemical, microorganism, substance, or injury, etc., that leads to neurodegeneration. Examples of “neurodegenerative insults” include, but are not limited to, reperfusion injuries, protein aggregation (e.g., Alzheimer's or Parkinson associated proteins), reactions of free radicals, insufficient blood supply, glutamate excitotoxicity, and oxidative stress. Other neurodegenerative insults are known in the art.

As used herein the term “neurological disease”, “neurological disorder” or “neurological condition” refers to a disease of the brain, spine and nerves that connect them. There are more than 600 diseases of the nervous system, such as brain tumors, epilepsy, Parkinson's disease and stroke. Major types of neurological diseases, disorders or conditions include, but are not limited to, diseases caused by faulty genes (e.g., Huntington's disease and muscular dystrophy); problems with the way the nervous system develops (e.g., spina bifida); degenerative disease where nerve cells are damaged or die (e.g., Parkinson's disease or Alzheimer's disease); diseases of the blood vessels that supply the brain (e.g., stroke); injuries to the spinal cord or brain; seizure disorders (e.g., epilepsy); cancer (e.g., brain tumors); and infections (e.g., meningitis).

As used herein, the term “neuroprotective effect” refers to a compound or agent that has the effect of preserving neuronal structure and/or function. In the case of a neurodegenerative insult, the compound or agent provides for the relative preservation of neuronal integrity, such that the rate of loss of neural integrity is reduced in the presence of the compound or agent than without.

As used herein, the term, “nonessential amino acids” refers to amino acids that can be made by a subject and is therefore not essential to the subject's diet. For humans, there are 11 nonessential amino acids: alanine, arginine, asparagine, aspartic acid, cysteine, glutamic acid, glutamine, glycine, proline, serine, and tyrosine.

As used herein, the term “neural progenitor cells” or “NPCs” refers to cells capable of dividing a limited number of times and have the capacity to differentiate into a restricted repertoire of neuronal and glial cell types.

As used herein, the term “neurobasal media” refers to cell or organoid growing basal medium that is designed for long-term maintenance and maturation of substantially pure or pure pre-natal and embryonic neuronal cell populations without the need for an astrocyte feeder layer when supplemented. Neurobasal media is commercially available from a variety of vendors, including ThermoFisher Scientific, VWR, Sigma Aldrich, US Bio, and STEMCELL Technologies.

As used herein, the term “oscillatory waves” refers to rhythmic or repetitive patterns of neural activity.

As used herein, the term “pyramidally-shaped neurons” refers to a multipolar neuron that has a conic shaped cell body.

As used herein, the term “serum-free” refers to cell or organoid growing media that is animal component-free. Serum-free media has fewer undefined components than serum containing media and generally is lower in protein content than media which has supplemented with serum, such as fetal bovine serum.

As used herein, the term “stem cells” refers to an undifferentiated cell of a multicellular organism that is capable of giving rise to indefinitely more cells of the same type, and from which certain other kinds of cell arise by differentiation.

As used herein, the term “supplemented neurobasal media” refers to neurobasal media that has been supplemented with factors and growth agents that promote the survival, growth and differentiation of neuronal cells. Supplements for neurobasal media can be purchased from a variety of vendors, including B-27™ Plus, N-2 and GlutaMAX™ supplements from ThermoFisher Scientific, NeuroCult™ and STMdiff™ supplements from STEMCELL Technologies, GEM21 NeuroPlex™ and N2 NeuroPlex™ from Gemini Bio-Products, and NDiff™ supplements from Sigma Aldrich.

As used herein, the term “vehicle control” refers to substance that is used as a vehicle for a solution of the experimental compound or drug product, which is used alone and is administered in the same manner as it is used with the experimental compound or drug product. Typically, the “vehicle control” is innocuous and does not change the activity of the cells or functional cortical organoids disclosed herein.

The disclosure demonstrates the formation of small-scale functional electrophysiological networks in cortical organoids, similar to those observed in the developing brain. The generation of cerebral organoids from induced pluripotent stem cells (iPSCs) offers a three-dimensional framework to study the developing human brain. Organoids generated from induced pluripotent stem cells (iPSC) provide a scaled-down and three-dimensional model of the human brain, mimicking various developmental features at the cellular and molecular level. Despite recent advances in the understanding of their vast cellular diversity, there is no evidence that these organoids show complex and functional neural network activity that resembles early human brain formation. Therefore, researchers have not yet clearly determined whether organoids are a suitable model for studying brain activity.

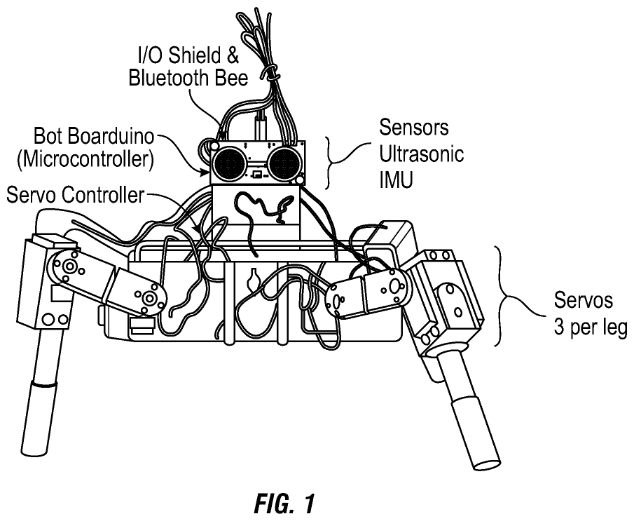

The disclosure demonstrates a method, system and device for using brain organoids to (a) control a device, (b) to assist in understanding basic neuronal development and neuronal feedback through a device and (c) to screen agents and neuronal interfaces for device control. The methods and systems of the disclosure use a brain organoid system that interfaces with a device to be controlled through an interface that can sense neuronal impulses. The neuronal impulses cause modulation of the device's activity through the interface. The interface can be connected to any number of different devices but is exemplified using a “spider robot” (see FIG. 1). In some embodiments, the device provides a feedback to the brain organoid. The feedback is typically performed through a second agent or physical delivery interface.

The methods and systems are useful for studying and teaching neuronal impulse modulation and neuronal development. For example, brain organoids can interact with a range of equipment: microchips and processors, robotic equipment (including motors, wheels, arms, etc.), cameras, and other hardware. Two-way functionality can also be used (electronic equipment sending signals to the organoid) such that those signals can be used to improve methods of connecting the organoid to the electronic circuitry (particularly the use of silicon probes connected to electronic micro-array). One example is medical devices that more seamlessly interface with the human body, and that a patient might be able to directly control (such as a spinal implant for those with spinal cord trauma, or a limb replacement for those who have lost an arm or leg, for example).

As demonstrated herein, the brain organoids of the disclosure provide for oscillatory waves across the culture. These waves can be measured by sensory arrays and the measured waves transformed to signals that direct a device to carry out a particular action. In some embodiments the organoids culture can be patterned such that the oscillatory waves bifurcate or oscillate in different patterns thus providing additional signals.

The disclosure describes the use of human iPSCs to generate an enriched pyramidal cortical population that self-assembles into functional network units over the span of several months. The method was designed (i) to provide a versatile organoid technology platform without the need for specialized equipment, (ii) to overcome structural and functional variability issues, (iii) to decrease the exposure to an excess of growth factors that would benefit for future disease modeling studies, and (iv) to develop a mature functional network system exhibiting EEG-like activity that would allow the direct interrogation of human cortical features. Further, investigations were directed to use of the organoids to study the molecular basis of human brain oscillatory activity formation, maintenance, and temporal control by targeting specific genes. Machine learning predictive analytics were applied to the organoids to evaluate the similarity between electrophysiological activity patterns of the in vitro model and human preterm neonatal electroencephalogram (EEG). Models based on organoids disclosed herein are suitable for investigating the physiological basis of network formation at early and late stages of the human brain development. The absence of spinning bioreactors, for instance, means that virtually any laboratory working with stem cells would be able to successfully generate thousands of cortical organoids, and this procedure would be efficient and at a low cost. Limiting the time of exposure to growth factors during neuronal maturation is especially advantageous when studying neurological disorders, since specific growth factors are able to rescue disease phenotypes. Needless to say, the ability to model the complex functional dynamics of the human brain for research and therapeutic purposes is paramount. The disclosure demonstrates that human neurons in vitro are able to generate neural activity comparable to in vivo human electrophysiology. These cortical organoid integrated models do not suffer from any the limitations seen with other brain cancer models (e.g., immune rejection), and can additionally be used in high-throughput put studies which are clearly not possible with other brain cancer models.

Pluripotent stem cells are a type of cell that undergoes self-renewal while maintaining an ability to give rise to all three-germ layer-derived tissues and germ cell lineages. Induced pluripotent stem cells are described by Shinya Yamanaka's team at Kyoto University, Japan. Yamanaka had identified genes that are particularly active in embryonic stem cells and used retroviruses to transfect mouse fibroblasts with a selection of those genes. Eventually, four key pluripotency genes essential for the production of pluripotent stem cells were isolated; Oct-3/4, SOX2, c-Myc, and Klf4. The same group published a study along with two other independent research groups from Harvard, MIT, and the University of California, Los Angeles, showing successful reprogramming of mouse fibroblasts into iPS and even producing a viable chimera. The methods of obtaining iPS cells are known and described in the literature (see, U.S. Pat. No. 9,005,966 and U.S. Pat. Publ. No. 2015/0159143A1, the disclosure of which are incorporated herein by reference). Briefly, terminally differentiated human fibroblast (e.g., human dermal fibroblasts) cells can be induced to de-differentiate. The disclosure contemplates the use of a variety of de-differentiation agents comprising KLF4, OCT4, SOX2, c-MYC or n-MYC, NANOG or any combination thereof (e.g., KLF4, OCT4, SOX2, c-MYC or n-MYC and optionally NANOG). Such de-differentiation agents include nucleic acids, peptides, polypeptides, small organic molecules, and antibodies that cause induction of any one or more of KLF4, OCT4, SOX2, c-MYC or n-MYC and NANOG. De-differentiation may be achieved by contacting a cell in vitro with one or more de-differentiation factors for a time sufficient to induce de-differentiation. In one aspect, the de-differentiation factors are transfected into a cell to be de-differentiated under the control of a constitutive or inducible promoter or as RNA replicons comprising multicistronic RNA molecule separated by cleavable peptides (e.g., 2A peptides and/or IRES domains).

Cell types that can be used in the methods and organoids of the disclosure include stem cells derived from any mammalian species including humans, monkeys, and apes and include embryonic stem cells, embryonic germ cells, and iPS cells (see, e.g., Nature, 448:313-318, July 2007; and Takahashi et al., Cell, 131(5):861-872; which are incorporated herein by reference). Stem cells can be induced to differentiate down a desired lineage using a number of techniques known in the art including, but not limited to, physical stimuli, chemical/biological factor stimuli (e.g., growth factors, media conditions), co-culturing techniques and any combination of the foregoing.

The method of the disclosure includes a number of steps including (i) inducing neural progenitor cells development from stem cells, (ii) expanding/proliferating the neuronal progenitor cells, (iii) inducing neural cell differentiation and (iv) stimulating neural cell maturation. For example, a general method includes neural induction of single-cell suspensions of feeder-free iPS cells through the inhibition of the bone morphogenetic protein (BMP) and transforming growth factor-β (TGF-β) pathways. During the stage of progenitor cell proliferation, developing cortical organoids were exposed to fibroblast and epidermal growth factors (FGF2 and EGF, respectively). The FGF2 and EGF were replaced by brain-derived neurotrophic factor (BDNF), glial cell-derived neurotrophic factor (GDNF) and neurotrophic factor 3 (NT3), in addition to ascorbic acid and dibutyryl-cAMP, to promote and accelerate neuronal differentiation. Maturation was completed in medium without the addition of growth factors. During this process, the newly formed cortical organoids grew in a robust and consistent manner.

As mentioned above, sources of stem cells useful in the disclosure are known and include induced pluripotent stem cells (iPSCs). Of particular advantage is that iPSCs can be obtained from subject with various neurological diseases and disorders cause by genetic abnormalities. Thus, iPSCs induced from cells from such subjects will contain the genetic abnormality. Moreover, cerebral organoids derived from such cells will include the abnormality. Such “abnormal” cerebral organoids can then be used to study the disease and screen for treatments. Additionally, iPSCs from normal patient can be gene edited to provide for “abnormal” cerebral organoids that can also be used to study and screen for treatments for neurological diseases, disorders or conditions. Examples of techniques for gene editing include, but are not limited to, gene therapy, meganuclease-based engineering, zinc finger nuclease-based engineering, TALEN, and CRISPR-Cas systems or the like.

The somatic cells used to obtain induced pluripotent stem cells can be isolated from any number of various tissues of the body. For example, cells may be obtained from bone marrow, fetal tissue (e.g., fetal liver tissue), peripheral blood, umbilical cord blood, pancreas, skin and the like. In one embodiment, the cells are fibroblasts and in a further embodiment, the cells are obtained from a subject having, or suspected of having, a mutation that causes a neurological disease or disorder. As is well known a somatic cell includes the genetic makeup of the individual and thus any induced pluripotent stem cell obtained from the somatic cell will include the same genetic makeup (e.g., same mutations found in the somatic cells obtained from the subject).

The methods of the disclosure may be applied to a procedure wherein differentiated (lineage committed) cells are removed from a subject, de-differentiated in culture, manipulated to re-differentiate along a specific differentiation pathway (e.g., neuronal cells) and then cultured and studied to (a) determine a mutations phenotypic result and/or (b) screen agents for their effect on the mutant dedifferentiated neuronal cell.

For example, fibroblasts can be removed from a subject, de-differentiated using de-differentiation factors (e.g., with a KLF4, OCT4, SOX2, c-MYC or n-MYC, NANOG agonists or any combination thereof) and optionally mitotically expanded and then differentiated with factors (including physical stimuli) known to cause differentiation of iPSCs down a lineage committed path. In one embodiment, the method comprises removing differentiated cells from an injured or diseased subject. In one embodiment, the “de-differentiated cells” are differentiated down a lineage committed path to study a particular disease. For example, the de-differentiated cells (e.g., iPSC) can be differentiated down a neuronal lineage to obtain cortical organoids.

The isolation of cells, such as fibroblasts, from a subject are known. For example, the isolation of fibroblasts may, for example, be carried out as follows: fresh tissue, e.g., a biopsy, samples are thoroughly washed and minced in Hanks balanced salt solution (HBSS) in order to remove serum. The minced tissue is incubated from 1-12 hours in a freshly prepared solution of a dissociating enzyme such as trypsin. After such incubation, the dissociated cells are suspended, pelleted by centrifugation and plated onto culture dishes. Fibroblasts cells will attach to the culture dish before other cells, therefore, appropriate stromal cells can be selectively isolated and grown.

Somatic cell (e.g., a population of somatic cells such as fibroblasts) are obtained from a subject are de-differentiated into induced pluripotent stem cells (iPSCs). For mutations having effects on neuronal processing and development, the iPSCs are then differentiated to neuronal cells (e.g., such as into cortical organoids). Because the genome of the iPSCs will carry any mutant gene present in the somatic cell, the differentiated neuronal cells will also carry the same mutation. In this way, the effect of the mutation on neuronal function can be studied. In addition, various factors or agents can be used to modulate the effect of the mutation on neuronal cell function, which may further be specific for the subject that was the source of the cells. In other words, the differentiated neuronal cells can be used to screen agents for effects on the biological function of the mutant neuronal cells. In this way, agents that show a beneficial effect on a particular mutation can then be advanced as potential therapeutics.

IPSCs can be cultured and expanded using number of known methods. In some embodiments the cells are cultured in a feeder free system. In yet another embodiment, the stem cells are culture in a feeder free animal free culture system (see, U.S. Pat. No. 8,609,417, which is incorporated herein by reference). The cultured stem cells (e.g., iPSCs) can be stored (i.e., “banked”) using commonly known techniques. For example, feeder-free iPSCs can be fed daily with mTeSR™1 media (STEMCELL Technologies, Seattle, Wash. U.S.A.).

In one embodiment, a pluripotent stem cell (e.g., an iPSC) is differentiated into a neural progenitor cell (NPC) using one or more factors (e.g., a SMAD inhibitor molecule). Exemplary embodiments include differentiating iPSC in the presence of SB431542. Alternative factors (individually and/or in combination) could be used in the disclosed methods. Though these factors are sometimes referred to as “dual” SMAD inhibitors, more or fewer than two factors may be utilized within the scope of these methods. Other SMAD inhibitors are known such as, but not limited to, dorsomorphin.

To induce neuronal-like stem cells, colonies of iPSCs are dissociated with Accutase (Life Technologies, Carlsbad, Calif., U.S.A.) in PBS (1:1) and resuspended in mTeSR™1 media supplemented with SB431542 and dosomorphin and cultured in the presence of a Rho kinase (ROCK) inhibitor under rotation and/or conditions to form free-floating spheres (sometimes referred to “neurospheres”). Exemplary ROCK inhibitors include Y-27632 (Calbiochem, Sigma-Aldrich, St. Louis, Mo., U.S.A.) and fasudil, which bind to the kinase domain to inhibit its enzymatic activity in an ATP-competitive mechanism. Negative regulators of ROCK activation include small GTP-binding proteins such as Gem, RhoE, and Rad, which can attenuate ROCK activity. H-1152 dihydrochloride (H-1152P-2HCl; (S)-(+)-2-Methyl-1-[(4-methyl-5-isoquinolinyl)sulfonyl] homopiperazine) can also be used. Additional ROCK inhibitors include those described in International Application Publication Nos.: WO 01/56988; WO 02/100833; WO 03/059913; WO 02/076976; WO 04/029045; WO 03/064397; WO 04/039796; WO 05/003101; WO 02/085909; WO 03/082808; WO 03/080610; WO 04/112719; WO 03/062225; and WO 03/062227, for example. In some of these cases, motifs in the inhibitors include an indazole core; a 2-aminopyridine/pyrimidine core; a 9-deazaguanine derivative; benzamide-comprising; aminofurazan-comprising; and/or a combination thereof. For example, WO 03/080610 relates to imidazopyridine derivatives as kinase inhibitors, such as ROCK inhibitors, and methods for inhibiting the effects of ROCK1 and/or ROCK2. The disclosures of the applications cited above are incorporated herein by reference.

After neurosphere formation, mTeSR media is replaced with Neurobasal (Life Technologies) supplemented with GlutaMAX™ (ThermoFisher Scientific), Gem21 NeuroPlex™ (Gemini Bio-Products, West Sacramento, Calif., U.S.A.), N2 NeuroPlex™ (Gemini Bio-Products), MEM nonessential amino acids (NEAA; Life Technologies), antibiotics (e.g., Pen/Strep), SB431542 and dorsomorphin and cultured for about 5-10 days (typically about 7 days). The cells are then maintained in Neurobasal (Life Technologies) supplemented with GlutaMAX™, Gem21 NeuroPlex™ (Gemini Bio-Products, West Sacramento, Calif., U.S.A.), MEM nonessential amino acids (NEAA; Life Technologies), antibiotics, and FGF2 for 5-10 days (typically about 7 days) to induce neuro-progenitor cell proliferation. This is followed by an additional 5-10 days (e.g., about 7 days) in Neurobasal (Life Technologies) supplemented with GlutaMAX™, Gem21 NeuroPlex™ (Gemini Bio-Products, West Sacramento, Calif., U.S.A.), MEM nonessential amino acids (NEAA; Life Technologies), antibiotics, FGF2 and EGF (PeproTech, Rocky Hill, N.J., U.S.A.).

International Application No. PCT/US2018/043983 describes methods of making cortical organoids (the disclosure PCT/US2018/043983 is incorporated herein by reference for all purposes). For example, following induction and proliferation of neuronal-progenitor cells, the cells are switch to media that promoted differentiation to cortical organoids. In one embodiment, the cells are cultured in media containing BDNF, GDNF and NT-3. For example, the cells are cultured with Neurobasal media (Life Technologies) supplemented with GlutaMAX™, Gem21 NeuroPlex™ (Gemini Bio-Products, West Sacramento, Calif., U.S.A.), MEM nonessential amino acids (NEAA; Life Technologies), antibiotics BDNF, GDNF, NT-3 (all from PeproTech), L-ascorbic acid and dibutyryl-cAMP. Following organoid development, the organoids can be maintained in Neurobasal (Life Technologies) supplemented with GlutaMAX™, Gem21 NeuroPlex™ (Gemini Bio-Products, West Sacramento, Calif., U.S.A.), MEM nonessential amino acids (NEAA; Life Technologies) and antibiotics, with media change ever 3-4 days.

For example, in the exemplary experiments described herein neural induction of single-cell suspensions of feeder-free iPS cells was achieved by the inhibition of the bone morphogenetic protein (BMP) and transforming growth factor-β (TGF-β) pathways. During progenitor cell proliferation, developing cortical organoids were exposed to fibroblast and epidermal growth factors (FGF2 and EGF, respectively). Next, FGF2 and EGF were replaced by brain-derived neurotrophic factor (BDNF), glial cell-derived neurotrophic factor (GDNF) and neurotrophic factor 3 (NT3), in addition to ascorbic acid and dibutyryl-cAMP, to promote and accelerate neuronal differentiation. Further maturation was completed in neural medium without the addition of growth factors. During this process, the newly formed cortical organoids grew in a robust and consistent manner.

While the functional difference between the organoids and a full neonatal cortex is notable, the current results represent the first step towards an in vitro model that captures the complex spatiotemporal oscillatory dynamics of the human brain. Robust extracellular electrical activity was established at earlier stages and progressively developed into an organized oscillatory network similar to that observed in human EEG. As such, it was shown that features of early functional network dynamics (e.g., spontaneous activity transients) can be recapitulated by an in vitro model of the developing cortex, with no additional constraints other than structural and genetic similarities. Organoid activity shows delta-high gamma phase-amplitude coupling during network-synchronous events, a hallmark of inter-regional cortical communication. Further, a differential role of glutamate and GABA in initiating and maintaining functional oscillatory network activity of the cortical organoids was accomplished. Additionally, the disappearance of oscillatory activity in developmental impaired organoids (MECP2-KO organoids) was also measured. Organoid network electrophysiological signatures spontaneously mimic human preterm neonatal EEG features between 28 and 38 post-conception weeks. This offers strong evidence for a convergent experience-independent neurodevelopmental program prior to birth. Given the potential roles of synchronized and oscillatory network dynamics in coordinating information flow between developed cortical brain regions during human cognition, these results highlight the potential for cortical organoids to advance the understanding of functional electrophysiology, brain development, and neuro-genetic disorders. The cortical organoids presented herein offer an innovative link between microscale organoid physiology and cognitive neuroscience.

Once the organoids has been generated the electrical (pulse) waves can be measured using various systems including multi-electrode arrays (MEAs) operably associated with the culture. The MEA can than transmit the impulse measurements to a computer that can transform the frequency and/or amplitude and/or direction of the wave into signals that can be used to control a mechanical or electrical device.

In one embodiment, a method or system of the disclosure includes: a sensor array for measuring electrophysiological responses and propagation waves of the brain organoid. The sensor array includes: a substrate; a multi-electrode array (MEA) disposed in or on the substrate; and optionally a plurality of interdigitized electrodes (IDEs) disposed in or on the substrate. The substrate can be a planar cell substrate or can be patterned or 3-dimensional substrate comprising brain organoid cells. The MEA and optionally the IDEs are interpenetrating within a plane substantially parallel to an upper surface of the substrate.

According to yet another general embodiment, a method of forming a system for simultaneously measuring electrophysiological responses and/or propagation waves of a brain organoid includes: forming a sensor array in or on a substrate surface.

More recently carbon nanotubes (CNTs) have been used in measuring electrophysiological responses. CNTs are well suited for neural electrical interfacing applications owing to their large surface area, superior electrical and mechanical properties. Over the past several years it has been demonstrated as a promising material for neural interfacing applications. It was shown that the CNTs coating enhanced both recording and electrical stimulation of neurons in culture, rats and monkeys by decreasing the electrode impedance and increasing charge transfer (Keefer, E. W. et al., 2008). Related work demonstrated the single-walled CNTs composite can serve as material foundation of neural electrodes with chemical structure better adapted with long-term integration with the neural tissue, which was tested on rabbit retinas, crayfish in vitro and rat cortex in vivo (Chen, Y.-C. et al., 2011; Gabriel, G. et al., 2009; Kam, N. W. S., Jan, E., & Kotov, N. A., 2008; Yi, W. et al., 2015).

The disclosure also provides a neural interface system comprising the brain organoid and multi-electrode array to transmit electronic information to a separate component through the use of a physical cable, including one or more of electrically conductive wires or optical fibers. Alternatively or additionally, transmission of data or other electronic information between discrete components can be accomplished wirelessly, by one or more discrete components including a transceiver that may transmit and receive data such as through the use of “Bluetooth” technology or according to any other type of wireless communication means, method, protocol or standard, including, for example, code division multiple access (CDMA), wireless application protocol (WAP), infrared or other optical telemetry, radiofrequency or other electromagnetic telemetry, ultrasonic telemetry, or other telemetric technology.

The method and system provides one or more sensors for detecting biological electrical waves in a brain organoid. As mentioned above the one or more sensors may include a plurality of electrodes that allow continual or chronic detection of the biological signals. A processing unit receives these signals from the sensor and utilizes various signal processing, electronic, mathematic, neural net and other techniques and processes to produce a processed signal used to control a device such as a prosthetic limb, ambulation vehicle, communication device, robot, computer or other controllable device. The system includes two or more discrete components, such as those defined by a housing or other enclosing or partially enclosing structure, or those defined as being detached or detachable from another discrete component. The discrete components of the system in their entirety include the one or more sensors (e.g., an MEA), the processing unit and the controlled device. Any one of the one or more sensors, the processing unit and the controlled device may be only partially included in a single discrete component, and a portion of one may be included with a portion or the entirety of another in a single discrete component.

Any and all discrete components may be in direct contact with the brain organoid or may be external (e.g., attached to a tissue culture plate or similar culture substrate). Discrete components can include include but are not limited to: a multiarray sensor (e.g., MEA), a processing unit, a controlled device, a display monitor, a calibration or system configuration module, a memory storage device, a telemetry device, a physical cable connecting device, a power supply module, a recharging module, an information recall and display unit, and a system diagnostic unit. In the instance where a discrete component includes a configuration module, the configuration module may include configuration programs, settings, or system specific data. In those instances, all data for a specific single system is associated, or electronically linked, with that system's unique electronic identifier. The configuration module uses the embedded unique electronic identifier during the configuration process to assure the proper data is utilized.

Electronic information or data is transmitted between one or more discrete components using one or more physical cables and/or wireless communication means. A unique electronic identifier, such as a unique alphanumeric code or serial number associated with the system, is included in one or more transmissions of electronic information between discrete components or between any discrete component and a separate device outside the system. Any and all communications that include the unique electronic identifier can be used to confirm that each discrete component is from the same or at least a compatible system. In wireless communication, the unique electronic identifier can be included in various handshaking protocols used in one or more information transmissions, such as handshaking protocols well known to those of skill in the art of wireless communication.

The various electrodes of the one or more sensors can detect signals, such as neuron spikes which emanate from the neurons brain organoid. The electrodes can take on various shapes and forms. The sensor can be placed on the surface of the brain organoid or on the surface of a substrate comprising the brain organoid to detect local field potential (LFP) signals, or to detect electroencephalogram (EEG) signals.

The sensor electrodes can be used to detect various signals including neuron spikes, electrocorticogram signals (ECoG), local field potential (LFP) signals, electroencelphalogram (EEG) signals and other signals. The electrodes can detect multicellular signals from clusters of neurons and provide signals midway between single neuron and electroencephalogram recordings. Each electrode is capable of recording a combination of signals, including a plurality of neuron spikes.

A processing unit receives the signals from the sensor and performs various signal processing functions including but not limited to amplification, filtering, sorting, conditioning, translating, interpreting, encoding, decoding, combining, extracting, mathematically transforming and/or otherwise processing those signals to generate a control signal for transmission to a controlled device. The processing unit may process signals that are mathematically combined, such as the combining neuron spikes that are first separated using spike discrimination methods known to those of skill in the art. The processing unit may include multiple components or a single component.

A controlled device is a computer system, other controlled devices can be included individually or in combination, including but not limited to prosthetic limbs, functional electrical stimulation (FES) devices and systems, robots and robotic components, teleoperated devices, computer controlled devices, communication devices, environmental control devices, vehicles such as wheelchairs, or remote control devices.

The sensor can be connected via a multi-conductor cable or wirelessly (e.g., through Bluetooth) to a processing unit. A multi-conductor cable can include a separate conductor for each electrode, as well as additional conductors to serve other purposes.

The processing unit can comprise various signal conditioning elements such as amplifiers, filters, and signal multiplexing circuitry.

The methods and systems described herein comprising the functional cortical organoids of the disclosure can be used, for example, to screen for the efficacy and/or cytotoxicity of compounds, growth/regulatory factors, pharmaceutical compounds, and the like on the stem cells or a particular lineage of cells derived/differentiated from the stem cells, to elucidate the mechanism of certain diseases by determining changes in the biological activity. The methods and systems described herein comprising the functional cortical organoids of the disclosure can be used to examine neurological diseases and disorders by generating neuronal cells from iPS cells obtained from somatic cells isolated from a subject having a neurological disease. The iPS cells are differentiated to neuronal cells and then the biological signaling and wave functions of such neurons to elucidate the mechanism of certain diseases by determining changes in the biological activity.

In addition, the methods and system of the disclosure can be used to screen and develop neural nets etc. that can be used to process information to control external devices. Such ability allows for the design of prosthetic limbs or other devices controller systems for assisted living of disabled persons.

The functional cortical organoids disclosed herein may be used to screen a wide variety of compounds, such as cytotoxic compounds, growth/regulatory factors, pharmaceutical agents, etc., or physical feedback system to modulate organoid function in controlling an external device. To this end, the functional cortical organoids disclosed herein are maintained and connected to a multi-component system comprising one or more sensors and processing devices that translate the organoids oscillatory waves to an external device (e.g., a robot). During “control” of the external device by the oscillatory waves, a feedback comprising physical or chemical stimuli (e.g., neurotransmitters etc.) can be provide to the cultured organoid and the resulting effect on oscillatory waves measured by changes in the activity of the external device (e.g., a robot).

The functional methods and systems of the disclosure comprising the cortical organoids and one or more external systems may be used as model systems for the study of physiologic or pathologic conditions.

Depending upon the intended use for the functional cortical organoids disclosed herein, various specialized cells or biological agents may be cultured with the cortical organoids. It should be noted, that since there is no immune rejection, there is no requirement that the cells that are cultured with the cortical organoids be from a certain genetic background or be from a certain species. It is envisaged herein, that additional specialized cells or biological agents can be used to model other disease or disorders, in particular neurodegenerative diseases and disorders, like Parkinson's disease and Alzheimer's disease.

The following examples are intended to illustrate but not limit the disclosure. While they are typical of those that might be used, other procedures known to those skilled in the art may alternatively be used.

### EXAMPLES

Cell source. iPSC lines derived from control individuals have been previously characterized elsewhere. Human embryonic stem cell (ESC) and iPSC colonies were expanded on Matrigel-coated dishes (BD Biosciences, San Jose, Calif., USA) with mTeSR1 medium (StemCell Technologies, Vancouver, Canada). The cells were routinely checked by karyotype and CNV arrays to avoid genomic alterations in the culture.

Teratoma formation. iPSC colonies were dissociated, re-suspended in PBS-Matrigel, and injected subcutaneously in NOD SCID mice. The tumor was dissected, fixed in and paraffin embedded after 8 weeks. Sections of 10 μm thickness were stained with hematoxylin and eosin and analyzed for the presence of the three germ layer tissues.

MECP2-KO cell line generation. MECP2-deficient cell lines were generated by inducing pluripotency in fibroblasts derived from a male patient. Additionally, H9 human ESC with the CRISPR/Cas9 genome-editing system were used to induce frameshift mutations in the MECP2 locus. This incorporation resulted in the creation of early stop codons rendering a non-functional MECP2 protein. Mutagenesis and off-targets were confirmed by exome sequencing techniques. The CRISPR-Cas protocol was as described in Thomas et al. (Cell Stem Cell (2017)). Once the pluripotency state of the cellular models was confirmed, they were differentiated into 2D neuronal monolayer cultures and cortical organoids.

Tissue source. Human fetal brain tissue was obtained under a protocol approved by the Human Research Protections Program Committee of the UCSD Institutional Review Board. All patients provided informed consent for collection and use of these tissues.

Generation of cortical organoids. Feeder-free iPSCs were fed daily with mTeSR1 for 7 days. Colonies were dissociated using Accutase (Life Technologies, Carlsbad, Calif., USA) in PBS (1:1) for 10-20 minutes at 37° C. and centrifuged for 3 minutes at 100×g. The cell pellet was re-suspended in mTeSR1 supplemented with 10 μM SB431542 (SB; Stemgent, Cambridge, Mass., USA) and 1 μM Dorsomorphin (Dorso; R&D Systems, Minneapolis, Minn., USA). Approximately 4×10⁶ cells were transferred to one well of a 6-well plate and kept in suspension under rotation (95 rpm) in the presence of 5 μM ROCK inhibitor (Y-27632; Calbiochem, Sigma-Aldrich, St. Louis, Mo., USA) for 24 hours to form free-floating spheres. After 2 or 3 days, mTeSR was substituted by Medial [Neurobasal (Life Technologies) supplemented with GlutaMAX™, 2% Gem21 NeuroPlex™ (Gemini Bio-Products, West Sacramento, Calif., USA), 1% N2 NeuroPlex™ (Gemini Bio-Products), 1% MEM nonessential amino acids (NEAA; Life Technologies), 1% penicillin/streptomycin (PS; Life Technologies), 10 μM SB and 1 μM Dorso] for 7 days. Then, the cells were maintained in Media2 [Neurobasal with GlutaMAX™, 2% Gem21 NeuroPlex™, 1% NEAA and 1% PS] supplemented with 20 ng/mL FGF2 (Life Technologies) for 7 days, followed by 7 additional days in Media2 supplemented with 20 ng/mL of FGF2 and 20 ng/mL EGF (PeproTech, Rocky Hill, N.J., USA). Next, cells were transferred to Media3 [Media2 supplemented with 10 μg/mL of BDNF, 10 μg/mL of GDNF, 10 μg/mL of NT-3 (all from PeproTech), 200 μM L-ascorbic acid and 1 mM dibutyryl-cAMP (Sigma-Aldrich)]. After 7 days, cortical organoids were maintained in Media2 for as long as needed, with media changes every 3-4 days.

Multi-electrode array (MEA). Six-week-old cortical organoids were plated per well in 12-well MEA plates (Axion Biosystems, Atlanta, Ga., USA). Each well contains 64 platinum microelectrodes with 30 μm of diameter spaced by 200 μm, yielding a total of 512 channels. The plate was previously coated with 100 μg/mL poly-L-ornithine and 10 μg/ml laminin, and four independent experiments were performed in duplicates. Cells were fed twice a week and measurements were collected 24 hours after the medium was changed, once a week, starting at two weeks after plating (8 weeks of organoid differentiation). Recordings were performed using a Maestro MEA system and AxIS software (Axion Biosystems) with a customized script for high band-pass filter (0.1-Hz and 5-kHz cutoff frequencies). Spikes were detected with AxIS software using an adaptive threshold set to 5.5 times the standard deviation of the estimated noise for each electrode (channel). The plate was first allowed to rest for three minutes in the Maestro device, and then four minutes of data were recorded. For the MEA analysis, the electrodes that detected at least 5 spikes/min were classified as active electrodes using Axion Biosystems' Neural Metrics Tool. Bursts were identified in the data recorded from each individual electrode using an inter-spike interval (ISI) threshold requiring a minimum number of 5 spikes with a maximum ISI of 100 ms. A minimum of 10 spikes under the same ISI with a minimum of 25% active electrodes were required for network bursts in the well. The synchrony index was calculated using a cross-correlogram synchrony window of 20 ms. Bright-field images were captured from each well to assess for neural density and electrode coverage over time.

Custom MEA analysis. Raw MEA recordings were converted to .mat files using Axion-provided functions and analyzed offline using custom MATLAB functions and scripts. Local field potential signals (LFP) from each of the 64 electrodes were generated by low-pass filtering (FIR filter) and downsampling raw signals from 12,500 Hz to 1,000 Hz (resample.m). Spikes were detected as follows: each channel was first referenced to the well median (64 channels). The median was used instead of the mean to avoid biasing the reference during high firing rate periods. Next, the re-referenced signal was bandpass filtered for 300-3,000 Hz with a 3rd-order Butterworth filter (butter.m). The spike threshold was set to be 5.5 standard deviations, where the standard deviation was estimated as previously described in Quiroga et al., Neural Comput. 16, 1661-87 (2004) to avoid biasing the threshold for channels with high firing rates (thus an artificially high threshold). Spike timestamps were taken as the peak time after the absolute value of the signal crossed the threshold, but at least 1 ms from another spike (findpeaks.m). Spike timestamps were then converted into binary vectors (1 ms bin size), summed across 64 channels, and smoothed (conv.m) with a normalized 100-point Gaussian window (gausswin.m) to create a population spiking vector for each MEA well. Note that spikes from each channel do not represent single-unit spikes as the spatial resolution of MEA electrodes were too sparse. Multi-unit spiking were not sorted since total population spiking (of well) was submitted for further analysis, rather than individual spike trains.

Network event analysis. A network event was detected when population spiking was (i) greater than 80% of the maximum spiking value over the length of the recording, (ii) at least 1 spike/s, and (iii) 1 second away from any other network events. The first peak after all 3 criteria were satisfied was marked as t=0, and the window of data, from 0.5 s before to 2.5 s after the peak was collected as the network event. LFP data from all 64 channels from the same timeframe were also collected for analysis. All events from different MEA wells obtained on the same recording day were aggregated for statistical analysis and plotting. Subpeaks within an event were identified using findpeaks.m, where a subpeak must satisfy the following: (i) peak height of at least 25% of the first peak, (ii) peak width of at least 50 ms, (iii) at least 200 ms away from the previous peak, and (iv) peak prominence of 1 over Peak 1 height. Subpeak time and the height relative to the initial peak were recorded. The inter-event interval coefficient of variation (IEI CV) was calculated as the standard deviation of the inter-event interval divided by its mean, where IEI is the time between consecutive network events within the same MEA well. Event temporal correlation was calculated as the mean Pearson correlation coefficient of population spiking vector during each network event with every other network event in the same MEA well across a single recording session. Event spatial correlation was calculated as the mean Pearson correlation coefficient between all pairs of 64 LFP channels during each 3-s network event.

Oscillatory spectral power analysis. Power spectral density (PSD) estimates were computed using Welch's method (pwelch.m), with a window length of 2 s and overlap of 1 s. Oscillatory power was defined as peaks in the PSD above the aperiodic 1/f power law decay. Thus, for each channel, a straight line was fit over the PSD in double-log space between 0.5-20 Hz using robust fit (robustfit.m), and oscillatory power was computed as the difference between the mean log PSD power and the mean log fitted power (baseline), over 2.5-4.5 Hz.

Phase Amplitude Coupling (PAC). LFP data from all 64 channels of each well was first lowpass/bandpass filtered (eegfilt.m, EEGLAB) for delta (0-4 Hz) and high-frequency, broadband gamma (200-400 Hz) activity. Delta phase was extracted by taking the phase angle of the bandpassed delta signal Hilbert transform (hilbert.m, angle.m), while gamma power was extracted by taking the squared magnitude of the filtered gamma. Gamma power was smoothed with the same delta-band filter for display purposes, but not for subsequent analysis. To compute PAC, instantaneous delta phase was binned into 20 equidistant bins between −Π and Π, and gamma power was sorted based on the corresponding delta phase at the same sample time and averaged across the same phase bin. This procedure was performed separately for event and non-event indices, where event indices are the same 3-s windows as described above in Network Event Analysis. Well-average gamma was calculated by aligning the binned gamma vector for each channel such that the phase of maximum gamma power was −Π and was averaged across all 64 channels. The resultant well-averaged gamma power was normalized and PAC was calculated as the Kullback-Leibler divergence between the distribution of gamma power across phase bins and a uniform distribution (also known as Modulation Index).

Pharmacology. The pharmacological manipulation was performed using the following drugs: 10 μM bicuculline, 50 μM muscimol, 20 μM CNQX, 20 μM AP5, 25 μM baclofen and 1 μM TTX. In this assessment, baseline recordings were obtained immediately before and 15 min after the addition of the compound. Three washes with PBS for total removal of the drug were performed in washout experiments; fresh media was added and another recording was conducted after 2 hours.

Preterm neonatal EEG. The dataset includes 567 recordings from 39 preterm neonates (24-38 weeks old conception age), consisting of 23 EEG features computed from the entirety of each recording, as well as during “low-activity periods” (46 features in total), and the post-conception age in weeks.

Statistical analysis for organoids. Data are presented as mean±s.e.m., unless otherwise indicated, and it was obtained from different samples. No statistical method was used to predetermine the sample size, and no adjustments were made for multiple comparisons. The statistical analyses were performed using Prism software (GraphPad, San Diego, Calif., USA). Student's t-test, Mann-Whitney-test, or ANOVA with post hoc tests were used as indicated. Significance was defined as P<0.05(*), P<0.01(**), or P<0.001(***). Blinding was used for comparing affected and control samples.

Emergence of complex oscillatory network activity. Neuronal activity was first evaluated by live calcium imaging, measured directly in the whole organoid. Neurons in the intact organoid showed a higher frequency of spontaneous calcium transients compared to neurons in 2D monolayer cultures. Single-cell neural activity was evaluated by live calcium imaging in 6-week-old organoids. The neurons showed sparse activity with a higher frequency of spontaneous calcium transients compared to monolayer cultures. Concurrently, weekly extracellular recordings of spontaneous electrical activity using multi-electrode arrays (MEA) were performed. Single-channel and population (whole-well, n=8) firing characteristics derived from channel-wise spike times were separately analyzed, as well as the local field potential (LFP); a measure of aggregate synaptic currents and other slow ionic exchanges. Over the course of 10 months, organoids exhibited consistent increases in electrical activity, as parametrized by channel-wise firing rate, burst frequency, and spike synchrony, which indicates a continually-maturing neural network. Organoid firing rates were far higher than previously observed in studies using iPSC-derived neurons or brain organoids and were ultimately comparable to rodent and primate brain activity. Additionally, the variability between replicates over 40 weeks of differentiation was significantly lower compared to iPSC-derived neurons in monolayer cultures.

Next, the population-level signals, typically observed in in vivo electrophysiology, were observed to further probe the functional network properties of the cortical organoid cultures. Specifically, in addition to the total network population firing rate derived from channel-wise spike times, the LFP was analyzed, a measure of aggregate synaptic currents and other slow ionic exchanges. During individual recordings, cultures displayed robust patterns of activity, switching between long periods of quiescence and short bursts of spontaneous network-synchronized spiking (hereafter referred to as “network events”).

From 4-months onwards, a secondary peak emerged 300-500 ms after the initial network activation, leading to the presence of a fast oscillatory (2-3 Hz) pattern up to 6-months in culture. The regular oscillatory activity during network events transitioned to stronger, yet more variable, oscillations over time. To quantify this network complexity, the regularity (coefficient of variation, CV) and the spatial and temporal correlation between spontaneous network events were tracked. The inter-event interval CV consistently increased over 10 months of differentiation, from extremely regular latencies (CV≅0) at 2 months to irregular, Poisson-like (CV≅1) at 10 months. This indicates increased variability between consecutive network events initiation. Additionally, spatial and temporal irregularity on a shorter time-scale (within-event) also increased with development, suggesting a breakdown of deterministic population dynamics from the onset of network events.

Up to 25 weeks, the number of subpeaks steadily increased over time, along with higher amplitudes and more frequent occurrences of network events, denoting a strengthening of the oscillatory network. The primary peak that marks the onset of network events remained the time of maximum population firing and increased for up to 30 weeks. The subsequent peaks continued to increase in amplitude relative to the first peak during the course of development, indicating a continued recruitment of downstream neurons into the oscillatory network. Remarkably, at 4 months, consistent and self-similar oscillatory structures in both population firing and LFP were observed, which was robust across replicates.

Total and oscillatory activity increased in cortical organoids, which also displayed an augment in the complexity of network activity. After 6 months, robust and invariant oscillatory activity during network events transitioned to become stronger and more variable over time. The number of peaks in the oscillatory event decreased, but the amplitude of each peak continued to increase. As a measure of network complexity, the regularity of spontaneous network events (coefficient of variation, CV, of inter-event intervals) were tracked along with the spatial and temporal correlation between network events over time. The CV of the inter-event interval increased consistently, indicating greater variability of network event initiation. Across all pairs of the 64 LFP channels, the spatial event similarity (mean absolute value of the pair-wise channel correlation coefficient during network events) showed an initial increase for up to 24 weeks. This observation likely reflects the increased connectivity and physical growth of the network over the electrode array. However, the spatial similarity of the LFP decreased in the subsequent weeks, indicating that it was not simply a measure of increased extracellular ionic diffusion. Instead, this finding suggests spatially varying patterns of activity within the same network event.

When comparing the population spiking waveform of one event to another (temporal similarity) within the same recording, a remarkable consistency during early development was observed. Namely, each time a network event occurred, it occurred in an extremely similar way. Unlike spatial similarity, the temporal similarity of the firing pattern reliably decreased with development, suggesting a breakdown of deterministic dynamics from the onset of network events. Taken together, the electrophysiological data revealed the development of the cortical organoid cultures across different network states: from a simple network with extreme rigidity and regularity, to one that acquires repetitive, perhaps overly-regular oscillatory patterns, until it finally reaches a stage of higher spatiotemporal complexity and variability that is more similar to the network observed in developed cortical tissue.

Based on the detection of slow extracellular signals in the LFP, network-level dynamics in the organoids to human brain tissue in vitro and in vivo were compared. At this stage, the organoid LFP does not exhibit the full temporal complexity observed in human electrocorticography, but notable resemblances do exist. In particular, EEG from extremely preterm infants (<30 months conceptional age) has been shown to display an electrophysiological signature known as trace discontinu, where quiescent periods are punctuated by brief high-amplitude fluctuations in the 1-4 Hz range. These quiescent periods quickly disappear as the child ages (with the exception of “burst suppression” during general anesthesia), but brief bursts of oscillatory activity are still a prominent feature in adult electrophysiology and are distinctly visible in the data trace. Similar bouts of bursting activity were observed between silent periods in a culture of human fetal brain tissue. Additionally, the time-frequency representation of network events in organoids resembled that of oscillatory bursts in fetal tissue and adult ECoG traces, with power localized to the low frequencies and often concentrating within a narrow oscillatory band.

Periodic oscillatory activity is often defined as a “bump” over the characteristic 1/f background noise in the power spectral density (PSD) of extracellular signals above-and-beyond the aperiodic 1/f signal. In organoid LFPs, both prominent oscillatory peaks in the low-frequency range (1-4 Hz), as well as the aperiodic signal characteristic of neural recordings were observed. The development of oscillatory activity in cortical organoids over time was quantified by computing the PSD for each LFP recording. Oscillatory power in the delta range (1-4 Hz) increased for up to 24 weeks in culture, tapering off slightly in subsequent recordings and plateauing during the last 10 weeks. This inverted-U trajectory reflects the network's initial acquisition of oscillatory modes at steady frequencies and the dispersion of this regularity at later time points. Using only event latency, event peak spiking, and oscillatory power, and their respective square roots, a linear regression model achieved an extremely good fit for individual organoids (R²=0.919±0.017, root mean square error (RMSE)=18.2±1.8 days, mean±s.e.m.). Furthermore, regression models trained on the same unnormalized features across all organoids achieved high prediction accuracy (R²=0.849±0.025, RMSE=41.3±6.1 days, leave-one-out cross-validation), indicating the consistent development of network electrophysiological features across organoids. The LFP results reveal the development of the cortical organoid cultures across different network states: from sparse activity with extreme rigidity and regularity, to one that acquires repetitive, perhaps overly-regular oscillatory patterns, until it finally reaches a stage of higher spatiotemporal complexity and variability that is more similar to the oscillatory networks observed in the human electrophysiology.

Oscillatory coordination of neural ensembles and its synaptic mechanisms. Oscillatory dynamics in the functioning brain have been postulated to coordinate spiking across neural ensembles. In the LFP and other mesoscopic brain signals, this manifest as a phenomenon known as cross-frequency phase-amplitude coupling (PAC), wherein the high-frequency content of the LFP is entrained to the phase of slow oscillations. PAC in the neocortex and hippocampus has been shown to be functionally relevant in a range of behaviors and neurological disorders. In the organoids, greater PAC between oscillatory delta (1-4 Hz) and broadband gamma activity (200-400 Hz) during network events was observed in comparison to quiescent periods. This result suggests that oscillations in the organoid mimic dynamics relevant for the intact brain and may serve as a model to understand the fundamental mechanisms behind the emergence of oscillatory networks in the developing human brain.

The role of glutamatergic and GABAergic synaptic transmission in forming oscillations by pharmacological intervention were further evaluated. Neuronal network activity in the absence of external stimulation suggests the presence of intrinsically active neurons in the cortical organoids. In order to evaluate the functionality of chemical synapses, 8-month-old organoids were exposed to selected compounds. Similar to primary cultures, the neuronal networks were susceptible to both glutamate receptor antagonists (AP5 and CNQX; NMDA and AMPA/kainate, respectively) and GABA receptor agonists (muscimol, GABAA; baclofen, GABAB) by significantly reducing the number of bursts, with a subsequent extinction of synchronous activity. In contrast, the GABA receptor antagonist bicuculline increased burst activity with no impact on synchrony indexes. The effect of pharmacological treatments on network activity was reversed after the drug washout. Neuronal electrical activity was blocked in the presence of tetrodotoxin (TTX). Notably, blockade of GABAergic transmission by bicuculline increased the number of network-synchronized events and did not affect peak population firing rates, but abolished oscillatory activity by erasing subsequent reverberant peaks. Therefore, the main excitatory and inhibitory neurotransmitter systems of the human cortex are present and involved in the establishment of a synchronized network in cortical organoids. Further, the data suggests that GABA transmission is crucial for the maintenance, but not the initiation of oscillatory activity. This is consistent with accounts of inhibition rhythmically coordinating pyramidal population's activity during early development.

The data demonstrate the in vitro electrophysiological dynamic formation in cortical organoids compared to the developed human brain. The current results suggest that cortical organoids represent a first step towards an in vitro model that captures the complex spatiotemporal and oscillatory dynamics of the human brain. Different from other studies, a robust neural activity was stablished at earlier stages and progressively culminate in a complex EEG-like oscillatory network. Given the potential roles of synchronized oscillations in coordinating the information flow between developed cortical brain regions during human cognition, these results illustrate the potential to advance future studies in functional electrophysiology, brain development, and genetic neural disorders by leveraging the natural, emergent development of complex neural activity in cortical organoids.

As described herein an organic network able to communicate with a robotic platform was developed by connecting human brain organoids directly to a robot using wireless information from multi-electrode arrays (MEA). By closing the gap between in vitro neurodevelopmental models and the brain, the study provides heretofore unique opportunities for investigating and manipulating the role of synchronous network activity in the developing human nervous system.

The system can work with any type of electronic/robotic platform. As a proof-of-principle a “spider-like robot” was used. The quadruped robotic platform processes external stimulus while simultaneously calculating the effective positions of each of its legs to maintain an upright posture and allow for fluid robot movements. In order to do so, the robot is designed with microcontrollers and other on-board processors, all Arduino-based, that are integrated with custom designed C++ libraries. The on-board processing enables the robot's ability to support fluid movements for current and future capabilities. These capabilities include: (1) receiving and processing directional commands, (2) incorporating inverse kinematic equations on-board to ensure the robot remains upright regardless of the direction or speed, and (3) adopting and integrating environmental sensors to enable further future autonomous capabilities.

The robotic platform has the ability to accept high-level commands from a neural interpretation application running on a remote computing system. These commands are sent through Bluetooth to the microcontroller and subsequently interpreted on-board. Currently, the external commands consist of both direction and speed inputs that are used for determining the effective motor positions and associated robot movement speeds (e.g., how fast the robot will move with corresponding direction). The robot can support 3-axis of movement along both the x-y plane in addition to height commands on the z-axis.

The microcontrollers incorporate inverse kinematic equations to allow for efficient and fluid movements when calculating each of the 12 motor (servo) positions simultaneously. To ensure the quadruped remains upright these equations are executed on-board the microcontroller and reflect various factors (e.g. height, direction, and speed) to dynamically balance the robot and continually maintain the center of gravity. The equations input the coordinates for where the body should generally move, along with the coordinates for where each individual leg should move. In other words, the inverse kinematic equations can be split into two portions, the leg inverse kinematics and the body inverse kinematics. All of these then undergo some basic conditioning for added fluidity and balance.

The robotic platform also integrates different sensors to support detecting and extracting environmental information. Currently, the robot uses an Ultrasonic sensor and an IMU sensor for external input. The Ultrasonic sensor relays its distance data to the microcontroller, which when combined with on-board processing, is capable of detecting objects within 25 centimeters and stopping robot movement. The system also supports sending the Ultrasonic sensor output stream of distance data back to the remote computer. The current Ultrasonic sensor has a range of 2 cm (0.8 inches) to 3 meters.

A gyro and accelerometer have also been integrated into the robotic platform. Currently, using the gyro, the robot stops when it is tilted over a certain threshold. And, similar to the Ultrasonic sensor, this data stream can be sent back to the remote computing system. The accelerometer is currently being used to ensure the robot is traveling at an appropriate speed. If necessary, it will also compensate for any discrepancies and adjust its speed as necessary.

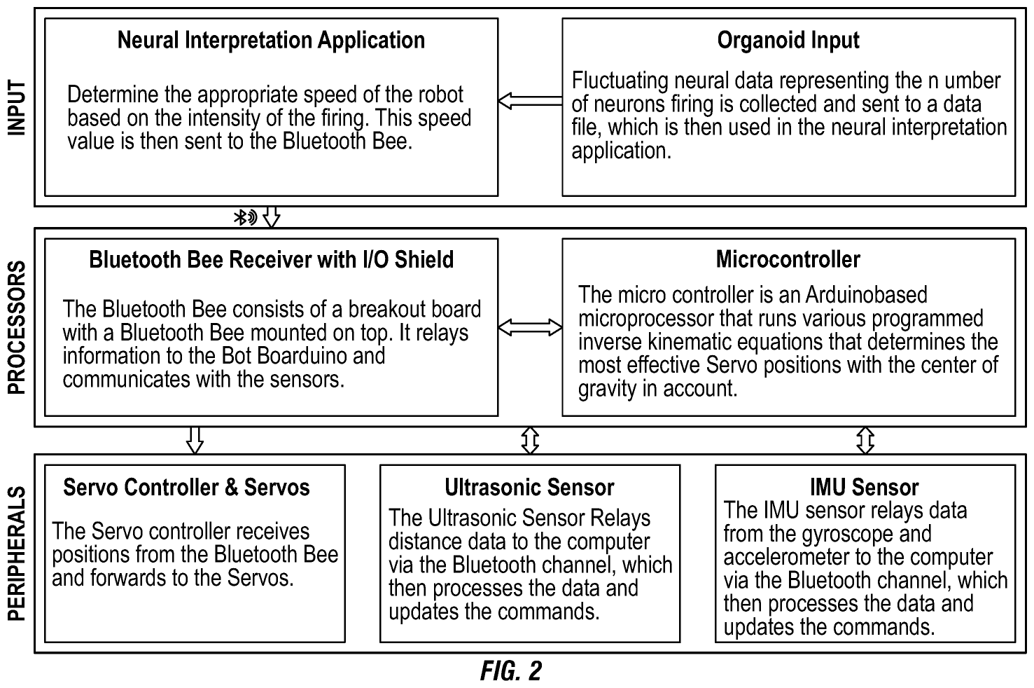

The overall system is depicted in FIG. 1. The external data is sent to the Neural Interpretation Application, which then samples and interprets the data to determine a corresponding speed. This speed and a command speed input is then sent to the I/O shield, which then relays it to the microcontroller. The microcontroller uses inverse kinematic equations to subsequently calculate effective Servo positions for the robot based on the command speed input. From there, the Servo (motors) positions are passed onto an SSC-32 (servo controller) that then relays this information to the individual Servos themselves. The sensors are connected to either the I/O shield or the microcontroller, and these boards process the sensor values and adjust the commands sent to the Servo controller, as necessary, FIG. 2. For example, once the Ultrasonic sensor comes within a certain distance threshold, the robot stops in place. These sensor values can also be fed back to the computing system for establishing a closed-loop system (e.g., feeding back to the computing system to cause delivery of a stimuli (e.g., physical, optical or chemical) to the brain organoid culture (e.g., to deliver GABA or other compound to modulate oscillatory signals in the culture).

Stimulation of external electric fields can be used to casually manipulate the effect of spontaneous and experience-independent network activity on neural development. For example, exogenously applied electric fields can entrain both spike timing and oscillations in the local field potential (LFP) of ferret cortical slices. The stimulation can also be achieved using optogenetics or transient exposure to ectopic neurotransmitters. Using the human organoid model, one can intervene and survey at a critical period where early and self-regulated network formation is ongoing.

With a physical robot, one can independently control the degrees of freedom present in a subject's sensorimotor system, as well as the physical environment it is embedded in, in order to study whether and how behaviorally-relevant adaptations can occur and alter the organoid circuits.

Initial work has translated recordings of organoid network activity into physical motion variables of a preliminary robotic embodiment, communicated through Bluetooth from a laptop. Additional work looks at an electrophysiological recording and processing setup that is capable of streaming and transforming spiking and LFP data in real-time. Further, a reverse connection can be provided to translate sensor information from the robot to stimulation patterns for the organoid—all in real-time (FIG. 4).

A number of embodiments have been described herein. Nevertheless, it will be understood that various modifications may be made without departing from the spirit and scope of this disclosure. Accordingly, other embodiments are within the scope of the following claims.

1. A brain-organoid machine interface, comprising:
a brain organoid culture;
a sensor comprising a plurality of electrodes that sense voltage change information including amplitude and/or frequency in the brain organoid culture;
a processing unit to receive the voltage change information from the sensor, process the voltage change information to produce processed signals, and transmit the processed signals; and
a controlled device for receiving the processed signals.

2. The brain-organoid machine interface of claim 1, wherein the sensor comprises a multi-electrode array (MEA) to sense voltage change information including amplitude and/or frequency in the brain organoid culture.

3. The brain-organoid machine interface of claim 1, wherein the sensor is attached to or immediately adjacent to a substrate comprising the brain organoid culture.

4. The brain-organoid machine interface of claim 1, wherein the sensor is in contact with the brain organoid culture.

5. The brain-organoid machine interface of claim 1, wherein the sensor and processing unit are connected with one or more cables comprising electrical or optically conducting wires.

6. The brain-organoid machine interface of claim 1, wherein the sensor and processing unit are wirelessly connected.

7. The brain-organoid machine interface of claim 1, wherein the voltage change information comprises one or more of neuron spikes, electrocorticogram signals, local field potential signals, and electroencephalogram signals.

8. The brain-organoid machine interface of claim 2, wherein the MEA is patterned.

9. The brain-organoid machine interface of claim 1, wherein the controlled device is one or more of the group consisting of a computer, a computer display, a mouse, a cursor, an artificial or prosthetic limb, a robot or robotic device, a computer controlled device, a vehicle, and a communication device or system.

10. The brain-organoid machine interface of claim 9, wherein the controlled device comprises a feed-back system.

11. The brain-organoid machine interface of claim 10, wherein the feed-back system comprises signals to deliver a physical, optical or chemical stimuli to the brain organoid culture transmitted from a sensor on the controlled device.

12. The brain-organoid machine interface of claim 1, wherein the brain organoid culture is comprised predominantly of proliferative neural progenitor cells (NPCs) that have self-organized into a polarized neuroepithelium-like structure.

13. The brain-organoid machine interface of claim 1, wherein the brain organoid culture comprise pyramidally-shaped neurons, dendritic spines and structurally defined synapses.

14. The brain-organoid machine interface of claim 1, wherein the brain organoid culture comprises cells differentiated from induced pluripotent stem cells (iPSCs).

15. The brain-organoid machine interface of claim 14, wherein the iPSCs are derived from somatic cells of a subject with a neurodegenerative disease or disorder.

16. The brain-organoid machine interface of claim 14, wherein the controlled device responds to the processed signals.

17. The brain-organoid machine interface of claim 16, wherein the responses of the controlled device are used to evaluate neurological activity and/or function of the brain organoid.

18. The brain-organoid machine interface of claim 17, wherein the controlled device is used to study changes in neurological activity and/or function in the presence and absence of an external stimuli.

19. The brain-organoid machine interface of claim 18, wherein the external stimuli is a chemical agent, drug or protein.
